SEQUENCE LISTING (138 sequences)

SEQ ID NO: 1 - protein, 118 aa, Artificial sequence
  Synthetic sequence: Amino acid sequence of the anti-ED-A antibody H1 heavy chain (VH)
EVQLVESGGGLVQPGGSLRLSCAASGFTFSPRRMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 2 - protein, 108 aa, Artificial sequence
  Synthetic sequence: Amino acid sequence of the anti-ED-A antibody H1 light chain (VL)
EIVLTQSPGTLSLSPGEKATLSCRASQSVSSAWLAWYQQKPGQAPRLLIYGASSRATGIPDRFSGSGSGTDFTLTISRLEPEDFAVYYCQQMRGRPPTFGQGTKVEIK

SEQ ID NO: 3 - protein, 3 aa, Artificial sequence
  Synthetic sequence: Amino acid sequence of the heavy chain CDR1 of anti-ED-A antibody H1
PRR

SEQ ID NO: 4 - protein, 6 aa, Artificial sequence
  Synthetic sequence: Amino acid sequence of the heavy chain CDR2 of anti-ED-A antibody H1
SGSGGS

SEQ ID NO: 5 - protein, 6 aa, Artificial sequence
  Synthetic sequence: Amino acid sequence of the heavy chain CDR3 of anti-ED-A antibody H1
STHLYL

SEQ ID NO: 6 - protein, 3 aa, Artificial sequence
  Synthetic sequence: Amino acid sequence of the light chain CDR1 of anti-ED-A antibody H1
SAW

SEQ ID NO: 7 - protein, 7 aa, Artificial sequence
  Synthetic sequence: Amino acid sequence of the light chain CDR2 of anti-ED-A antibody H1
GASSRAT

SEQ ID NO: 8 - protein, 6 aa, Artificial sequence
  Synthetic sequence: Amino acid sequence of the light chain CDR3 of anti-ED-A antibody H1
MRGRPP

SEQ ID NO: 9 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 133 aa, Artificial sequence [sequence omitted]

SEQ ID NO: 11 - protein, 20 aa, Artificial sequence
  Synthetic sequence: Amino acid sequence of the anti-ED-A antibody H1 linker sequence
GGGGSGGGGSGGGGSGGGGS

SEQ ID NO: 12 - DNA, 354 bp, Artificial sequence
  Synthetic sequence: Nucleotide sequence of the anti-ED-A antibody H1 heavy chain (VH)
gaggtgcagctggtggagtctgggggaggcttggtacagcctggggggtccctgagactctcctgtgcagcctctggattcacctttagcccgcggaggatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagt

SEQ ID NO: 13 - DNA, 324 bp, Artificial sequence
  Synthetic sequence: Nucleotide sequence of the anti-ED-A antibody H1 light chain (VL)
gaaattgtgttgacgcagtctccaggcaccctgtctttgtctccaggggaaaaagccaccctctcctgcagggccagtcagagtgttagctctgcgtggttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaa

SEQ ID NO: 14 - DNA, 60 bp, Artificial sequence
  Synthetic sequence: Nucleotide sequence of the anti-ED-A antibody H1 linker sequence
ggcggtggaggttctggcggcggtggcagtggcggtggaggttccgggggtggaggatct

SEQ ID NO: 15 - DNA, 354 bp, Artificial sequence
  Synthetic sequence: Nucleotide sequence of the F8-IL2 heavy chain (VH)
gaggtgcagctggtggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctcgagt

SEQ ID NO: 16 - DNA, 15 bp, Artificial sequence
  Synthetic sequence: Nucleotide sequence of the linker linking the VH and VL domains of F8 in the F8-IL2 conjugate
ggcggtagcggaggg

SEQ ID NO: 17 - DNA, 324 bp, Artificial sequence
  Synthetic sequence: Nucleotide sequence of the F8-IL2 light chain (VL)
gaaattgtgttgacgcagtctccaggcaccctgtctttgtctccaggggaaaaagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaa

SEQ ID NO: 18 - DNA, 45 bp, Artificial sequence
  Synthetic sequence: Nucleotide sequence of the linker linking the IL2 sequence to the F8 VL domain
tcttcctcatcgggtagtagctcttccggctcatcgtccagcggc

SEQ ID NO: 19 - DNA, 399 bp, Artificial sequence
  Synthetic sequence: IL2 nucleotide sequence
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 20 - skipped

SEQ ID NO: 21 - protein, 118 aa, Artificial sequence
  Synthetic sequence: Amino acid sequence of the anti-ED-A antibody B2 VH domain
EVQLVESGGGLVQPGGSLRLSCAASGFTFSAAKMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 22 - protein, 108 aa, Artificial sequence
  Synthetic sequence: Amino acid sequence of the anti-ED-A antibody B2 VL domain
EIVLTQSPGTLSLSPGEKATLSCRASQSVSVAFLAWYQQKPGQAPRLLIYGASSRATGIPDRFSGSGSGTDFTLTISRLEPEDFAVYYCQQMRGRPPTFGQGTKVEIK

SEQ ID NO: 23 - protein, 3 aa, Artificial sequence
  Synthetic sequence: Amino acid sequence of the heavy chain CDR1 of anti-ED-A antibody B2
AAK

SEQ ID NO: 24-25 - skipped (2 entries)

SEQ ID NO: 26 - protein, 3 aa, Artificial sequence
  Synthetic sequence: Amino acid sequence of the light chain CDR1 of anti-ED-A antibody B2
VAF

SEQ ID NO: 27 - skipped

SEQ ID NO: 28 - protein, 5 aa, Artificial sequence
  Synthetic sequence: Linker motif
SSSSG

SEQ ID NO: 29 - protein, 5 aa, Artificial sequence
  Synthetic sequence: Linker motif
GGGGS

SEQ ID NO: 30 - protein, 5 aa, Artificial sequence
  Synthetic sequence: Linker motif
GSGSA

SEQ ID NO: 31 - protein, 118 aa, Artificial sequence
  Synthetic sequence: Amino acid sequence of the anti-ED-A antibody G9 VH domain
EVQLVESGGGLVQPGGSLRLSCAASGFTFSHMQMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 32 - protein, 108 aa, Artificial sequence
  Synthetic sequence: Amino acid sequence of the anti-ED-A antibody G9 VL domain
EIVLTQSPGTLSLSPGEKATLSCRASQSVSTAFLAWYQQKPGQAPRLLIYGASSRATGIPDRFSGSGSGTDFTLTISRLEPEDFAVYYCQQMRGRPPTFGQGTKVEIK

SEQ ID NO: 33 - protein, 3 aa, Artificial sequence
  Synthetic sequence: Amino acid sequence of the heavy chain CDR1 of anti-ED-A antibody G9
HMQ

SEQ ID NO: 34-35 - skipped (2 entries)

SEQ ID NO: 36 - protein, 3 aa, Artificial sequence
  Synthetic sequence: Amino acid sequence of the light chain CDR1 of anti-ED-A antibody G9
TAF

SEQ ID NO: 37 - protein, 15 aa, Artificial sequence
  Synthetic sequence: Linker with three tandem repeats
SSSSGSSSSGSSSSG

SEQ ID NO: 38 - protein, 15 aa, Artificial sequence
  Synthetic sequence: Linker with three tandem repeats
GGGGSGGGGSGGGGS

SEQ ID NO: 39 - protein, 15 aa, Artificial sequence
  Synthetic sequence: Linker with three tandem repeats
GSGSAGSGSAGSGSA

SEQ ID NO: 40 - protein, 15 aa, Artificial sequence
  Synthetic sequence: Linker with three tandem repeats
GGSGGGGSGGGGSGG

SEQ ID NO: 41 - protein, 118 aa, Artificial sequence
  Synthetic sequence: Amino acid sequence of the anti-ED-A antibody C5 VH domain
EVQLVESGGGLVQPGGSLRLSCAASGFTFSPITMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 42 - protein, 108 aa, Artificial sequence
  Synthetic sequence: Amino acid sequence of the anti-ED-A antibody C5 VL domain
EIVLTQSPGTLSLSPGEKATLSCRASQSVSLHFLAWYQQKPGQAPRLLIYGASSRATGIPDRFSGSGSGTDFTLTISRLEPEDFAVYYCQQMRGRPPTFGQGTKVEIK

SEQ ID NO: 43 - protein, 3 aa, Artificial sequence
  Synthetic sequence: Amino acid sequence of the heavy chain CDR1 of anti-ED-A antibody C5
PIT

SEQ ID NO: 44-45 - skipped (2 entries)

SEQ ID NO: 46 - protein, 3 aa, Artificial sequence
  Synthetic sequence: Amino acid sequence of the light chain CDR1 of anti-ED-A antibody C5
LHF

SEQ ID NO: 47-50 - skipped (4 entries)

SEQ ID NO: 51 - protein, 118 aa, Artificial sequence
  Synthetic sequence: Amino acid sequence of the anti-ED-A antibody D5 VH domain
EVQLVESGGGLVQPGGSLRLSCAASGFTFSVMKMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 52 - protein, 108 aa, Artificial sequence
  Synthetic sequence: Amino acid sequence of the anti-ED-A antibody D5 VL domain
EIVLTQSPGTLSLSPGEKATLSCRASQSVSNAFLAWYQQKPGQAPRLLIYGASSRATGIPDRFSGSGSGTDFTLTISRLEPEDFAVYYCQQMRGRPPTFGQGTKVEIK

SEQ ID NO: 53 - protein, 3 aa, Artificial sequence
  Synthetic sequence: Amino acid sequence of the heavy chain CDR1 of anti-ED-A antibody D5
VMK

SEQ ID NO: 54-55 - skipped (2 entries)

SEQ ID NO: 56 - protein, 3 aa, Artificial sequence
  Synthetic sequence: Amino acid sequence of the light chain CDR1 of anti-ED-A antibody D5
NAF

SEQ ID NO: 57-60 - skipped (4 entries)

SEQ ID NO: 61 - protein, 118 aa, Artificial sequence
  Synthetic sequence: Amino acid sequence of the anti-ED-A antibody E5 VH domain
EVQLVESGGGLVQPGGSLRLSCAASGFTFSTGSMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 62 - protein, 108 aa, Artificial sequence
  Synthetic sequence: Amino acid sequence of the anti-ED-A antibody E5 VL domain
EIVLTQSPGTLSLSPGEKATLSCRASQSVSLAHLAWYQQKPGQAPRLLIYGASSRATGIPDRFSGSGSGTDFTLTISRLEPEDFAVYYCQQMRGRPPTFGQGTKVEIK

SEQ ID NO: 63 - protein, 3 aa, Artificial sequence
  Synthetic sequence: Amino acid sequence of the heavy chain CDR1 of anti-ED-A antibody E5
TGS

SEQ ID NO: 64-65 - skipped (2 entries)

SEQ ID NO: 66 - protein, 3 aa, Artificial sequence
  Synthetic sequence: Amino acid sequence of the light chain CDR1 of anti-ED-A antibody E5
LAH

SEQ ID NO: 67-70 - skipped (4 entries)

SEQ ID NO: 71 - protein, 118 aa, Artificial sequence
  Synthetic sequence: Amino acid sequence of the anti-ED-A antibody C8 VH domain
EVQLVESGGGLVQPGGSLRLSCAASGFTFSLQTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 72 - protein, 108 aa, Artificial sequence
  Synthetic sequence: Amino acid sequence of the anti-ED-A antibody C8 VL domain
EIVLTQSPGTLSLSPGEKATLSCRASQSVSLPFLAWYQQKPGQAPRLLIYGASSRATGIPDRFSGSGSGTDFTLTISRLEPEDFAVYYCQQMRGRPPTFGQGTKVEIK

SEQ ID NO: 73 - protein, 3 aa, Artificial sequence
  Synthetic sequence: Amino acid sequence of the heavy chain CDR1 of anti-ED-A antibody C8
LQT

SEQ ID NO: 74-75 - skipped (2 entries)

SEQ ID NO: 76 - protein, 3 aa, Artificial sequence
  Synthetic sequence: Amino acid sequence of the light chain CDR1 of anti-ED-A antibody C8
LPF

SEQ ID NO: 77-80 - skipped (4 entries)

SEQ ID NO: 81 - protein, 118 aa, Artificial sequence
  Synthetic sequence: Amino acid sequence of the anti-ED-A antibody F8 heavy chain (VH)
EVQLVESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 82 - protein, 108 aa, Artificial sequence
  Synthetic sequence: Amino acid sequence of the anti-ED-A antibody F8 light chain (VL)
EIVLTQSPGTLSLSPGEKATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 83 - protein, 3 aa, Artificial sequence
  Synthetic sequence: Amino acid sequence of the heavy chain CDR1 of anti-ED-A antibody F8
LFT

SEQ ID NO: 84 - protein, 5 aa, Artificial sequence
  Synthetic sequence: Amino acid sequence of the linker linking the VH and VL domains of F8 in the F8-IL2 conjugate
GGSGG

SEQ ID NO: 85 - skipped

SEQ ID NO: 86 - protein, 3 aa, Artificial sequence
  Synthetic sequence: Amino acid sequence of the light chain CDR1 of anti-ED-A antibody F8
MPF

SEQ ID NO: 87-90 - skipped (4 entries)

SEQ ID NO: 91 - protein, 118 aa, Artificial sequence
  Synthetic sequence: Amino acid sequence of the anti-ED-A antibody F1 VH domain
EVQLVESGGGLVQPGGSLRLSCAASGFTFSQARMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 92 - protein, 108 aa, Artificial sequence
  Synthetic sequence: Amino acid sequence of the anti-ED-A antibody F1 VL domain
EIVLTQSPGTLSLSPGEKATLSCRASQSVSAPFLAWYQQKPGQAPRLLIYGASSRATGIPDRFSGSGSGTDFTLTISRLEPEDFAVYYCQQMRGRPPTFGQGTKVEIK

SEQ ID NO: 93 - protein, 3 aa, Artificial sequence
  Synthetic sequence: Amino acid sequence of the heavy chain CDR1 of anti-ED-A antibody F1
QAR

SEQ ID NO: 94-95 - skipped (2 entries)

SEQ ID NO: 96 - protein, 3 aa, Artificial sequence
  Synthetic sequence: Amino acid sequence of the light chain CDR1 of anti-ED-A antibody F1
APF

SEQ ID NO: 97-100 - skipped (4 entries)

SEQ ID NO: 101 - protein, 118 aa, Artificial sequence
  Synthetic sequence: Amino acid sequence of the anti-ED-A antibody B7 VH domain
EVQLVESGGGLVQPGGSLRLSCAASGFTFSHFDMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 102 - protein, 108 aa, Artificial sequence
  Synthetic sequence: Amino acid sequence of the anti-ED-A antibody B7 VL domain
EIVLTQSPGTLSLSPGEKATLSCRASQSVSLAFLAWYQQKPGQAPRLLIYGASSRATGIPDRFSGSGSGTDFTLTISRLEPEDFAVYYCQQMRGRPPTFGQGTKVEIK

SEQ ID NO: 103 - protein, 3 aa, Artificial sequence
  Synthetic sequence: Amino acid sequence of the heavy chain CDR1 of anti-ED-A antibody B7
HFD

SEQ ID NO: 104-105 - skipped (2 entries)

SEQ ID NO: 106 - protein, 3 aa, Artificial sequence
  Synthetic sequence: Amino acid sequence of the light chain CDR1 of anti-ED-A antibody B7
LAF

SEQ ID NO: 107-110 - skipped (4 entries)

SEQ ID NO: 111 - protein, 118 aa, Artificial sequence
  Synthetic sequence: Amino acid sequence of the anti-ED-A antibody E8 VH domain
EVQLVESGGGLVQPGGSLRLSCAASGFTFSDMHMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 112 - protein, 108 aa, Artificial sequence
  Synthetic sequence: Amino acid sequence of the anti-ED-A antibody E8 VL domain
EIVLTQSPGTLSLSPGEKATLSCRASQSVSSSFLAWYQQKPGQAPRLLIYGASSRATGIPDRFSGSGSGTDFTLTISRLEPEDFAVYYCQQMRGRPPTFGQGTKVEIK

SEQ ID NO: 113 - protein, 3 aa, Artificial sequence
  Synthetic sequence: Amino acid sequence of the heavy chain CDR1 of anti-ED-A antibody E8
DMH

SEQ ID NO: 114-115 - skipped (2 entries)

SEQ ID NO: 116 - protein, 3 aa, Artificial sequence
  Synthetic sequence: Amino acid sequence of the light chain CDR1 of anti-ED-A antibody E8
SSF

SEQ ID NO: 117 - protein, 118 aa, Artificial sequence
  Synthetic sequence: VH domain of anti-ED-A antibody F8
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 118 - protein, 118 aa, Artificial sequence
  Synthetic sequence: VH domain of anti-ED-A antibody H1
EVQLLESGGGLVQPGGSLRLSCAASGFTFSPRRMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 119 - protein, 118 aa, Artificial sequence
  Synthetic sequence: VH domain of anti-ED-A antibody B2
EVQLLESGGGLVQPGGSLRLSCAASGFTFSAAKMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 120 - protein, 118 aa, Artificial sequence
  Synthetic sequence: VH domain of anti-ED-A antibody C5
EVQLLESGGGLVQPGGSLRLSCAASGFTFSPITMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 121 - protein, 118 aa, Artificial sequence
  Synthetic sequence: VH domain of anti-ED-A antibody D5
EVQLLESGGGLVQPGGSLRLSCAASGFTFSVMKMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 122 - protein, 118 aa, Artificial sequence
  Synthetic sequence: VH domain of anti-ED-A antibody E5
EVQLLESGGGLVQPGGSLRLSCAASGFTFSTGSMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 123 - protein, 118 aa, Artificial sequence
  Synthetic sequence: VH domain of anti-ED-A antibody C8
EVQLLESGGGLVQPGGSLRLSCAASGFTFSLQTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 124 - protein, 118 aa, Artificial sequence
  Synthetic sequence: VH domain of anti-ED-A antibody F1
EVQLLESGGGLVQPGGSLRLSCAASGFTFSQARMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 125 - protein, 118 aa, Artificial sequence
  Synthetic sequence: VH domain of anti-ED-A antibody B7
EVQLLESGGGLVQPGGSLRLSCAASGFTFSHFDMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 126 - protein, 118 aa, Artificial sequence
  Synthetic sequence: VH domain of anti-ED-A antibody E8
EVQLLESGGGLVQPGGSLRLSCAASGFTFSDMHMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 127 - protein, 118 aa, Artificial sequence
  Synthetic sequence: VH domain of anti-ED-A antibody G9
EVQLLESGGGLVQPGGSLRLSCAASGFTFSHMQMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 128 - protein, 108 aa, Artificial sequence
  Synthetic sequence: VL domain of anti-ED-A antibody F8
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 129 - protein, 108 aa, Artificial sequence
  Synthetic sequence: VL domain of anti-ED-A antibody H1
EIVLTQSPGTLSLSPGERATLSCRASQSVSSAWLAWYQQKPGQAPRLLIYGASSRATGIPDRFSGSGSGTDFTLTISRLEPEDFAVYYCQQMRGRPPTFGQGTKVEIK

SEQ ID NO: 130 - protein, 108 aa, Artificial sequence
  Synthetic sequence: VL domain of anti-ED-A antibody B2
EIVLTQSPGTLSLSPGERATLSCRASQSVSVAFLAWYQQKPGQAPRLLIYGASSRATGIPDRFSGSGSGTDFTLTISRLEPEDFAVYYCQQMRGRPPTFGQGTKVEIK

SEQ ID NO: 131 - protein, 108 aa, Artificial sequence
  Synthetic sequence: VL domain of anti-ED-A antibody C5
EIVLTQSPGTLSLSPGERATLSCRASQSVSLHFLAWYQQKPGQAPRLLIYGASSRATGIPDRFSGSGSGTDFTLTISRLEPEDFAVYYCQQMRGRPPTFGQGTKVEIK

SEQ ID NO: 132 - protein, 108 aa, Artificial sequence
  Synthetic sequence: VL domain of anti-ED-A antibody D5
EIVLTQSPGTLSLSPGERATLSCRASQSVSNAFLAWYQQKPGQAPRLLIYGASSRATGIPDRFSGSGSGTDFTLTISRLEPEDFAVYYCQQMRGRPPTFGQGTKVEIK

SEQ ID NO: 133 - protein, 108 aa, Artificial sequence
  Synthetic sequence: VL domain of anti-ED-A antibody E5
EIVLTQSPGTLSLSPGERATLSCRASQSVSLAHLAWYQQKPGQAPRLLIYGASSRATGIPDRFSGSGSGTDFTLTISRLEPEDFAVYYCQQMRGRPPTFGQGTKVEIK

SEQ ID NO: 134 - protein, 108 aa, Artificial sequence
  Synthetic sequence: VL domain of anti-ED-A antibody C8
EIVLTQSPGTLSLSPGERATLSCRASQSVSLPFLAWYQQKPGQAPRLLIYGASSRATGIPDRFSGSGSGTDFTLTISRLEPEDFAVYYCQQMRGRPPTFGQGTKVEIK

SEQ ID NO: 135 - protein, 108 aa, Artificial sequence
  Synthetic sequence: VL domain of anti-ED-A antibody F1
EIVLTQSPGTLSLSPGERATLSCRASQSVSAPFLAWYQQKPGQAPRLLIYGASSRATGIPDRFSGSGSGTDFTLTISRLEPEDFAVYYCQQMRGRPPTFGQGTKVEIK

SEQ ID NO: 136 - protein, 108 aa, Artificial sequence
  Synthetic sequence: VL domain of anti-ED-A antibody B7
EIVLTQSPGTLSLSPGERATLSCRASQSVSLAFLAWYQQKPGQAPRLLIYGASSRATGIPDRFSGSGSGTDFTLTISRLEPEDFAVYYCQQMRGRPPTFGQGTKVEIK

SEQ ID NO: 137 - protein, 108 aa, Artificial sequence
  Synthetic sequence: VL domain of anti-ED-A antibody E8
EIVLTQSPGTLSLSPGERATLSCRASQSVSSSFLAWYQQKPGQAPRLLIYGASSRATGIPDRFSGSGSGTDFTLTISRLEPEDFAVYYCQQMRGRPPTFGQGTKVEIK

SEQ ID NO: 138 - protein, 108 aa, Artificial sequence
  Synthetic sequence: VL domain of anti-ED-A antibody G9
EIVLTQSPGTLSLSPGERATLSCRASQSVSTAFLAWYQQKPGQAPRLLIYGASSRATGIPDRFSGSGSGTDFTLTISRLEPEDFAVYYCQQMRGRPPTFGQGTKVEIK

(19) United States
(12) Patent Application Publication
(10) Pub. No.: US 2011/0318302 A1
(43) Pub. Date: Dec. 29, 2011

(54) Cancer Treatment

(76) Inventors: Kathrin Schwager, Zurich (CH); Katharina Frey, Kandel (DE); Christoph Schliemann, Zurich (CH); Neri Dario, Buchs (CH)

(21) Appl. No.: 13/140,150

(22) PCT Filed: Dec. 23, 2009
(86) PCT No.: PCT/EP2009/009251

§ 371 Date: Sep. 6, 2011

Related U.S. Application Data
(60) Provisional application No. 61/142,974, filed on Jan. 7, 2009.

(51) Int. Cl. A61K 39/395; A61P 35/00

(52) U.S. Cl. 424/85.2

(57) ABSTRACT

The invention relates to the treatment of cancer, e.g. kidney cancer, using chemotherapeutic agents and immunocytokines, in particular immunocytokines which bind to the Extra Domain-A (ED-A) isoform of fibronectin.

The present invention relates to the treatment of cancer using chemotherapeutic agents and immunocytokines, in particular immunocytokines which bind to the Extra Domain-A (ED-A) isoform of fibronectin.

Fibronectin (FN) is a glycoprotein and is widely expressed in a variety of normal tissues and body fluids. It is a component of the extracellular matrix (ECM), and plays a role in many biological processes, including cellular adhesion, cellular migration, haemostasis, thrombosis, wound healing, tissue differentiation and oncogenic transformation.

Different FN isoforms are generated by alternative splicing of three regions (ED-A, ED-B, IIICS) of the primary transcript FN pre mRNA, a process that is modulated by cytokines and extracellular pH (Balza et al., 1988; Carnemolla et al., 1989; Borsi et al., 1990; Borsi et al., 1995). Expression of the ED-A isoform of fibronectin has been reported in a number of different cancers including kidney cancer, breast cancer, liver cancer, fibrosarcoma, rhabdomyosarcoma and melanoma (Lohi et al. 1995, Jacobs et al. 2002, Matsumoto et al. 1999, Oyama et al. 1989, Tavian et al. 1994, Borsi et al. 1987).

Antibodies specific for the ED-A isoform of fibronectin have been described previously and have been shown to efficiently target the tumour neovasculature in vivo (Villa et al., 2008).

The present inventors have discovered that antibody-cytokine conjugates which bind the ED-A isoform of fibronectin exhibit a surprising synergy with anti-cancer compounds such as sunitinib in the treatment of cancer.

Thus, an aspect of the invention provides a method of treating cancer comprising:
administering an anti-cancer compound and an antibody-cytokine conjugate to an individual in need thereof,
wherein the antibody-cytokine conjugate comprises a cytokine, e.g. interleukin 2 (IL2), conjugated to an antibody which specifically binds the Extra Domain-A (ED-A) isoform of fibronectin.

Treatment of cancer, as referred to herein, may refer to providing an anti-tumour effect. Thus, a method of treating cancer may be a method of providing an anti-tumour effect.

Other aspects of the invention provide an anti-cancer compound for use in a method of treating cancer comprising administering an anti-cancer compound and an antibody-cytokine conjugate comprising a cytokine, e.g. interleukin 2 (IL2), conjugated to an antibody which specifically binds to the ED-A isoform of fibronectin to an individual in need thereof and the use of an anti-cancer compound in the manufacture of a medicament for treatment of cancer, wherein the treatment comprises administering the anti-cancer compound and an antibody-cytokine conjugate to an individual in need thereof,
said antibody-cytokine conjugate comprising a cytokine, e.g. interleukin 2 (IL2), conjugated to an antibody which specifically binds to the ED-A isoform of fibronectin.

Other aspects of the invention provide an antibody-cytokine conjugate comprising a cytokine, e.g. interleukin 2 (IL2), conjugated to an antibody which specifically binds to the ED-A isoform of fibronectin for use in a method of treating cancer comprising administering the antibody-cytokine conjugate and an anti-cancer compound to an individual in need thereof and the use of an antibody-cytokine conjugate comprising a cytokine, e.g. interleukin 2 (IL2), conjugated to an antibody which specifically binds to the ED-A isoform of fibronectin in the manufacture of a medicament for treatment of cancer, wherein the treatment comprises administering the antibody-cytokine conjugate and the anti-cancer compound to an individual in need thereof.

Other aspects of the invention provide a combination of an anti-cancer compound and an antibody-IL2 conjugate comprising interleukin 2 (IL2) conjugated to an antibody which specifically binds to the ED-A isoform of fibronectin for use in a method of treating cancer comprising administering the antibody-IL2 conjugate and the anti-cancer compound to an individual in need thereof and the use of a combination of an anti-cancer compound and an antibody-IL2 conjugate comprising interleukin 2 (IL2) conjugated to an antibody which specifically binds to the ED-A isoform of fibronectin in the manufacture of a medicament for treatment of cancer comprising administering the antibody-IL2 conjugate and the anti-cancer compound to an individual in need thereof.

Cancers suitable for treatment as described herein include any type of solid or non-solid cancer or malignant lymphoma and especially liver cancer, lymphoma, leukaemia, sarcomas, skin cancer, bladder cancer, breast cancer, uterine cancer, ovarian cancer, prostate cancer, lung cancer, colorectal cancer, cervical cancer, head and neck cancer, oesophageal cancer, pancreatic cancer, renal cancer, stomach cancer and cerebral cancer. Cancers may be familial or sporadic. Cancers may be metastatic or non-metastatic. Preferably, the cancer expresses the ED-A isoform of fibronectin and/or is susceptible to sunitinib treatment.

Preferably, the cancer is a cancer selected from the group of kidney cancer, breast cancer, liver cancer, lung cancer, lymphoma, sarcoma (e.g. gastrointestinal stromal tumor), skin cancer (e.g. melanoma), colorectal cancer, and neuroendocrine tumours.

In some preferred embodiments, the cancer may be kidney cancer.

Anti-cancer compounds are cytotoxic compounds which inhibit the growth, division and/or proliferation of cancer cells. Anti-cancer compounds may, in some circumstances, have an effect on normal non-cancer cells in a patient. An anti-cancer compound may, for example, be a receptor tyrosine kinase inhibitor.

A receptor tyrosine kinase inhibitor is a chemotherapeutic compound which inhibits the activity of one or more receptor tyrosine kinases. Many suitable receptor tyrosine kinase inhibitors compounds are known in the art for use in the treatment of cancer, including, for example, sunitinib (marketed as Sutent), Erlotinib hydrochloride (marketed as Tarceva), Gefitinib (marketed as Iressa), and lapatinib ditosylate (Tykerb). Tyrosine kinase inhibitors may be used as described herein in any convenient form or formulation. For example, any suitable isomer, salt, solvate, chemically protected form, or prodrug of a particular tyrosine kinase inhibitor may be employed.

In some preferred embodiments, the tyrosine kinase inhibitor is sunitinib (N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide). Sunitinib is marketed under the trade name Sutent and has been approved for the treatment of renal cell carcinoma and imatinib-resistant gastrointestinal stromal tumor. In addition, sunitinib is also being investigated for the treatment of other cancers, such as breast cancer, lung cancer, colorectal cancer, and neuroendocrine tumors.

An antibody-IL2 conjugate for use as described herein may comprise interleukin 2 (IL2) conjugated to an antibody which specifically binds to the ED-A isoform of fibronectin.

Interleukin-2 (IL2) is a secreted cytokine which is involved in immunoregulation and the proliferation of T and B lymphocytes. IL2 has been shown to have a cytotoxic effect on tumour cells and recombinant human IL2 (aldesleukin: Proleukin®) has FDA approval for treatment of metastatic renal carcinoma and metastatic melanoma. The sequence of human IL2 is set out in SEQ ID NO: 9 below and publicly available under sequence database reference NP_{—}000577.2 GI: 28178861.

(hIL2 precursor sequence
[mature hIL2: residues 7-150]
SEQ ID NO: 9
MYRMQLLSCI ALSLALVTNS APTSSSTKKT QLQLEHLLLD
LQMILNGINN YKNPKLTRML TFKFYMPKKA TELKHLQCLE
EELKPLEEVL NLAQSKNFHL RPRDLISNIN VIVLELKGSE
TTFMCEYADE TATIVEFLNR WITFCQSIIS TLT

In some preferred embodiments, the IL2 moiety of the antibody-IL2 conjugate comprises a sequence which has at least 90% sequence identity, at least 95% sequence identity or at least 98% sequence identity to the mature human IL2 sequence set out in SEQ ID NO: 9.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol. Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

The IL2 moiety of the antibody-IL2 conjugate may comprise the sequence of mature human IL2 set out in SEQ ID NO: 9. In some especially preferred embodiments, the IL2 moiety of the antibody-IL2 conjugate comprises the sequence shown in FIG. 7E (i.e. amino acids 21-153 of human IL2 set out in SEQ ID NO: 9).

The IL2 moiety may be fused upstream (N-terminal) or downstream (C-terminal) of the antibody or polypeptide component thereof.

The IL2 moiety may be connected or attached to the antibody moiety of the antibody-IL2 conjugate by any suitable covalent or non-covalent means. In preferred embodiments, the antibody-IL2 conjugate may be a fusion protein comprising IL2 and an antibody which specifically binds to the ED-A isoform of fibronectin or a polypeptide component thereof (e.g. a heavy chain or a light chain of an antibody or multi-chain antibody fragment, such as a Fab). Thus, for example, the IL2 moiety may be fused to a VH domain or VL domain of the antibody. Typically the antibody, or component thereof, and IL2 moiety are joined via a peptide linker, e.g. a peptide of about 5-25 residues, e.g. 10-20 residues, preferably about 15 residues. Suitable examples of peptide linkers are well known in the art. In some embodiments, a linker may have an amino acid sequence as set out in SEQ ID NO: 37. Normally, the linker has an amino acid sequence comprising one or more tandem repeats of a motif. Typically the motif is a five residue sequence, and preferably at least 4 of the residues are Gly or Ser. Where four of the five residues is Gly or Ser, the other residue may be Ala. More preferably each of the five residues is Gly or Ser. Preferred motifs are SSSSG, GGGGS, GSGSA and GGSGG. Preferably, the motifs are adjacent in the sequence, with no intervening residues between the repeats. The linker sequence may comprise or consist of between one and five, preferably three or four, most preferably three repeats of the motif. For example, a linker with three tandem repeats may have one of the following amino acid sequences:

| | |
|---|---|
| SSSSGSSSSGSSSSG | SEQ ID NO: 37 |
| GGGGSGGGGSGGGGS | SEQ ID NO: 38 |
| GSGSAGSGSAGSGSA | SEQ ID NO: 39 |
| GGSGGGGSGGGGSGG | SEQ ID NO: 40 |

In preferred embodiments, the antibody moiety of the antibody-IL2 conjugate specifically binds to ED-A of fibronectin.

Preferred antibodies are tumour specific and bind preferentially to tumour tissue relative to normal tissue. Antibodies may, for example, bind to the neovascular structures of tumour tissue preferentially to normal tissue.

Examples of suitable antibodies for use in antibody-IL2 conjugates are disclosed in WO2008/120101 and Villa et al. (2008).

In some embodiments, the antibody moiety of an antibody-IL2 conjugate as described herein competes for binding to the ED-A isoform of fibronectin with an antibody comprising the F8 VH domain of SEQ ID NO. 81 and the F8 VL domain of SEQ ID NO. 82.

Competition between antibodies may be assayed easily in vitro, for example using ELISA and/or by tagging a specific reporter molecule to one antibody which can be detected in the presence of other untagged antibody(s), to enable identification of antibodies which bind the same epitope or an overlapping epitope.

The antibody moiety of the antibody-IL2 conjugate may bind the Extra Domain-A isoform of fibronectin (A-FN) and/or the ED-A of fibronectin with the same affinity as anti-ED-A antibody F8, H1, B2, C5, D5, E5, C8, F1, B7, E8 or G9, e.g. in scFv format, or with an affinity that is better. For example, antibody F8 binds the A-FN and the ED-A of fibronectin with a KD of 3×10−9 M. Preferably, a binding member for use in the invention binds the A-FN and/or the ED-A of fibronectin with the same affinity as antibody F8, or with an affinity that is better.

A suitable antibody for use in an antibody-IL2 conjugate as described herein may comprise one or more complementarity determining regions (CDRs) of antibody F8, H1, B2, C5, D5, E5, C8, F1, B7, E8 or G9, or variants thereof. Preferably, the antibody comprises one or more complementarity determining regions (CDRs) of antibody F8 or variants thereof.

A suitable antibody for use in an antibody-IL2 conjugate as described herein may comprise an antibody antigen binding site comprising a VH domain and a VL domain,
the VH domain comprising a VH CDR1 of SEQ ID NO: 3, 23, 33, 43, 53, 63, 73, 83, 93, 103 or 113, a VH CDR2 of SEQ ID NO. 4, and/or a VH CDR3 of SEQ ID NO. 5;
the VL domain comprising a VL CDR1 of SEQ ID NO: 6, 26, 36, 46, 56, 66, 76, 86, 96, 106 or 116, a VL CDR2 of SEQ ID NO. 7, and/or a VL CDR3 of SEQ ID NO. 8.

Preferably, the antibody for use in an antibody-IL2 conjugate as described herein comprises an antibody antigen binding site comprising a VH domain and a VL domain,
the VH domain comprising a VH CDR1 of SEQ ID NO: 83, a VH CDR2 of SEQ ID NO. 4, and/or a VH CDR3 of SEQ ID NO. 5;
the VL domain comprising a VL CDR1 of SEQ ID NO: 86, a VL CDR2 of SEQ ID NO. 7, and/or a VL CDR3 of SEQ ID NO. 8.

In some preferred embodiments, the antibody may comprise an antibody antigen binding site comprising the VH and the VL domain of any one of antibodies F8, H1, B2, C5, D5, E5, C8, F1, B7, E8 and G9. Most preferably, the antibody comprises an antibody antigen binding site comprising the F8 VH domain of SEQ ID NO. 81 and the F8 VL domain of SEQ ID NO. 82.

Variants of these VH and VL domains and CDRs may also be employed in antibodies for use in antibody-IL2 conjugates as described herein. Suitable variants can be obtained by means of methods of sequence alteration, or mutation, and screening.

Particular variants for use as described herein may include one or more amino acid sequence alterations (addition, deletion, substitution and/or insertion of an amino acid residue), maybe less than about 20 alterations, less than about 15 alterations, less than about 10 alterations or less than about 5 alterations, 4, 3, 2 or 1. Alterations may be made in one or more framework regions and/or one or more CDRs. In particular, alterations may be made in VH CDR1, VH CDR2 and/or VH CDR3, especially VH CDR3.

Preferably, a suitable variant for use as described herein comprises an antibody antigen binding site comprising a VH domain and a VL domain of any one of antibodies F8, H1, B2, C5, D5, E5, C8, F1, B7, E8 and G9, wherein the valine (V) residue at position 5 of the VH domain is substituted with leucine (L), and/or the lysine (K) residue at position 18 of the VL domain is substituted with arginine (R). Most preferably, a suitable variant for use as described herein comprises an antibody antigen binding site comprising the F8 VH domain of SEQ ID NO. 81 and the F8 VL domain of SEQ ID NO. 82, wherein the valine (V) residue at position 5 of the VH domain is substituted with leucine (L), and/or the lysine (K) residue at position 18 of the VL domain is substituted with arginine (R).

The nucleotide sequence of the F8-IL2 conjugate is shown in FIG. 6 and the amino acid sequence of the F8-IL2 conjugate is shown in FIG. 7.

The nucleotide sequence of anti-ED-A antibody H1 is shown in FIG. 8. The amino acid sequence of the anti-ED-A antibody H1 is shown in FIG. 9.

Preferred nucleotide sequences encoding VH and/or VL domains of anti-ED-A antibodies B2, C5, D5, E5, C8, F8, F1, B7, E8 and G9 are identical to nucleotide sequences encoding VH and/or VL domains of anti-ED-A antibody H1, except that the nucleotide sequences encoding the H1 CDR1s of the light (VL) and heavy (VH) chain are substituted with the nucleotide sequences encoding the light (VL) and heavy (VH) chain CDR1s listed in Table 1 for the respective antibody.

The amino acid sequences of the VH domains of anti-ED-A antibodies B2, C5, D5, E5, C8, F8, F1, B7, E8 and G9 are identical to the amino acid sequence of the H1 VH domain except that the H1 VH CDR1 is replaced by the VH CDR1 sequence listed in Table 1 for the relevant antibody. Similarly, the amino acid sequences of the VL domains of anti-ED-A antibodies B2, C5, D5, E5, C8, F8, F1, B7, E8 and G9 are identical to the amino acid sequence of the H1 VL domain except that the H1 VL CDR1 is replaced by the VL CDR1 sequence listed in Table 1 for the relevant antibody.

Administration of the anti-cancer compound, antibody-IL2 conjugate and compositions comprising one or both of these molecules is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage etc, is within the responsibility of general practitioners and other medical doctors.

The precise dose will depend upon a number of factors, the size and location of the area to be treated, the precise nature of the antibody-IL2 conjugate (e.g. whole antibody, fragment or diabody). A typical antibody-IL2 conjugate dose will be in the range 0.5 mg to 100 g for systemic applications, and 10 μg to 1 mg for local applications. Typically, the antibody moiety of the conjugate will be a whole antibody, preferably the IgG1 or IgG4 isotype. This is a dose for a single treatment of an adult patient, which may be proportionally adjusted for children and infants, and also adjusted for other antibody formats in proportion to molecular weight.

Appropriate doses and regimens for anti-cancer compounds are well known in the art.

Treatments may be repeated at daily, twice-weekly, weekly or monthly intervals, at the discretion of the physician.

The antibody-IL2 conjugate and the anti-cancer compound may be administered sequentially or simultaneously in accordance with any suitable regimen.

The antibody-IL2 conjugate and the anti-cancer compound will usually be administered to an individual in the form of pharmaceutical compositions, which may comprise at least one component in addition to the active compound.

Suitable components include a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

The antibody-IL2 conjugate and the anti-cancer compound may be formulated in separate pharmaceutical compositions or, where appropriate, in the same pharmaceutical composition.

Another aspect of the invention provides a pharmaceutical composition for use in the treatment of cancer comprising an anti-cancer compound and an antibody-IL2 conjugate comprising interleukin 2 (IL2) conjugated to an antibody which specifically binds to the ED-A isoform of fibronectin.

Another aspect of the invention provides a method of making a pharmaceutical composition for use in the treatment of cancer comprising formulating an anti-cancer compound and an antibody-IL2 conjugate comprising interleukin 2 (IL2) conjugated to an antibody which specifically binds to the ED-A isoform of fibronectin.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Another aspect of the invention provides a therapeutic kit for use in the treatment of cancer comprising an anti-cancer compound and an antibody-IL2 conjugate comprising interleukin 2 (IL2) conjugated to an antibody which specifically binds to the ED-A isoform of fibronectin.

The components of a kit (i.e. the anti-cancer compound and antibody-IL2 conjugate) are sterile and in sealed vials or other containers. A kit may further comprise instructions for use of the components in a method described herein. The components of the kit may be comprised or packaged in a container, for example a bag, box, jar, tin or blister pack.

### BRIEF DESCRIPTION OF THE DRAWINGS

FIG. 1: Treatment schedule of the two treatment intervals. FIG. 1 shows the treatment schedules for the two treatment intervals indicated in FIGS. 2 to 5. The numbers at the top of the figure refer to days post tumour implantation.

Treatment interval 1 extended over days 11-17 (post tumour implantation) and treatment interval 2 extended over days 37-43 (post tumour implantation). The different agents administered during the two treatment schedules are indicated on the left of the figure. Each arrow indicates treatment with a particular agent on a specific day post tumour implantation. Thus, for example, F8-IL2 was administered on days 11, 14 and 17 during treatment interval 1 and on days 37, 40 and 43 during treatment interval 2.

FIG. 2: Tumor growth curve—Sorafenib. FIG. 2 shows the tumour volume in mm³ in mice treated with PBS, F8-IL2, Sorafenib, or F8-IL2 and Sorafenib between 11 and 47 days after tumour implantation. “Treatment 1” and “Treatment 2” refer to the two treatment intervals, respectively. PBS, F8-IL2, Sorafenib, and F8-IL2 and Sorafenib were administered to the tumour-bearing mice during treatment intervals 1 and 2 in accordance with the schedule shown in FIG. 1.

FIG. 3: Tumor growth curve—Interferon alpha. FIG. 3 shows the tumour volume in mm³ in mice treated with PBS, F8-IL2, IFNa (interferon alpha), or F8-IL2 and IFNa between 11 and 47 days after tumour implantation. “Treatment 1” and “Treatment 2” refer to the two treatment intervals, respectively. PBS, F8-IL2, IFNa, and F8-IL2 and IFNa were administered to the tumour-bearing mice during treatment intervals 1 and 2 in accordance with the schedule shown in FIG. 1.

FIG. 4: Tumor growth curve—Sunitinib. FIG. 4 shows the tumour volume in mm³ in mice treated with PBS, F8-IL2, sunitinib, or F8-IL2 and sunitinib between 11 and 47 days after tumour implantation. “Treatment 1” and “Treatment 2” refer to the two treatment intervals, respectively. PBS, F8-IL2, sunitinib, and F8-IL2 and sunitinib were administered to the tumour-bearing mice during treatment intervals 1 and 2 in accordance with the schedule shown in FIG. 1.

FIG. 5: Tumor growth curve—Overview of all treatment groups. FIG. 5 summarizes the information already shown in FIGS. 2 to 4 and shows the tumour volume in mm³ in mice treated with PBS, Sorafenib, sunitinib, IFNa (interferon alpha), F8-IL2, F8-IL2 and Sorafenib, F8-IL2 and sunitinib, or F8-IL2 and IFNa.

FIG. 6: Nucleotide sequence of the F8-IL2 conjugate. FIG. 6 A: shows the nucleotide sequence of the F8-IL2 heavy chain (VH). The nucleotide sequence of the VH CDR1 is underlined. The nucleotide sequence of the VH CDR2 is shown in italics and underlined. The nucleotide sequence of the VH CDR3 is shown in bold and underlined. B: Shows the nucleotide sequence of the linker linking the VH and VL domains of F8 in the F8-IL2 conjugate. C: Shows the nucleotide sequence of the F8-IL2 light chain (VL). The nucleotide sequence of the VL CDR1 is underlined. The nucleotide sequence of the VL CDR2 is shown in italics and underlined. The nucleotide sequence of the VL CDR3 is shown in bold and underlined. D: Shows the nucleotide sequence of the linker linking the IL2 sequence to the F8 VL domain. E: Shows the IL2 nucleotide sequence.

FIG. 7: Amino acid sequence of the F8-IL2 conjugate. FIG. 7 A: shows the amino acid sequence of the anti-ED-A antibody F8 heavy chain (VH) (SEQ ID NO: 81). The amino acid sequence of the VH CDR1 (SEQ ID NO: 83) of anti-ED-A antibody F8 is underlined. The amino acid sequence of the VH CDR2 (SEQ ID NO: 4) of anti-ED-A antibody F8 is shown in italics and underlined. The amino acid sequence of the VH CDR3 (SEQ ID NO: 5) of anti-ED-A antibody F8 is shown in bold and underlined. B: Shows the amino acid sequence of the linker linking the VH and VL domains of F8 in the F8-IL2 conjugate (SEQ ID NO: 84). C: Shows the amino acid sequence of the anti-ED-A antibody F8 light chain (VL) (SEQ ID NO: 82). The amino acid sequence of the VL CDR1 (SEQ ID NO: 86) of anti-ED-A antibody F8 is underlined. The amino acid sequence of the VL CDR2 (SEQ ID NO: 7) of anti-ED-A antibody F8 is shown in italics and underlined. The amino acid sequence of the VL CDR3 (SEQ ID NO: 8) of anti-ED-A antibody F8 is shown in bold and underlined. D: Shows the amino acid sequence of the linker linking the IL2 sequence to the F8 VL domain. E: Shows the IL2 amino acid sequence.

FIG. 8: Shows the nucleotide sequence of the anti-ED-A antibody H1 heavy chain (VH) (SEQ ID NO: 12). The nucleotide sequence of the heavy chain CDR1 of anti-ED-A antibody H1 is underlined. The nucleotide sequence of the heavy chain CDR2 of the anti-ED-A antibody H1 is shown in italics and underlined. The nucleotide sequence of the heavy chain CDR3 of anti-ED-A antibody H1 is shown in bold and underlined. B: Shows the nucleotide sequence of the anti-ED-A antibody H1 linker sequence (SEQ ID NO: 14). C: Shows the nucleotide sequence of the anti-ED-A antibody H1 light chain (VL) (SEQ ID NO: 13). The nucleotide sequence of the light chain CDR1 of anti-ED-A antibody H1 is underlined. The nucleotide sequence of the light chain CDR2 of the anti-ED-A antibody H1 is shown in italics and underlined. The nucleotide sequence of the light chain CDR3 of anti-ED-A antibody H1 is shown in bold and underlined.

FIG. 9: Shows the amino acid sequence of the anti-ED-A antibody H1 heavy chain (VH) (SEQ ID NO: 1). The amino acid sequence of the heavy chain CDR1 (SEQ ID NO: 3) of anti-ED-A antibody H1 is underlined. The amino acid sequence of the heavy chain CDR2 (SEQ ID NO: 4) of the anti-ED-A antibody H1 is shown in italics and underlined. The amino acid sequence of the heavy chain CDR3 (SEQ ID NO: 5) of anti-ED-A antibody H1 is shown in bold and underlined. B: Shows the amino acid sequence of the anti-ED-A antibody H1 linker sequence (SEQ ID NO: 11). C: Shows the amino acid sequence of the anti-ED-A antibody H1 light chain (VL) (SEQ ID NO: 2). The amino acid sequence of the light chain CDR1 (SEQ ID NO: 6) of anti-ED-A antibody H1 is underlined. The amino acid sequence of the light chain CDR2 (SEQ ID NO: 7) of the anti-ED-A antibody H1 is shown in italics and underlined. The amino acid sequence of the light chain CDR3 (SEQ ID NO: 8) of anti-ED-A antibody H1 is shown in bold and underlined.

### TERMINOLOGY

### Antibody

This describes an immunoglobulin whether natural or partly or wholly synthetically produced. The term also covers any polypeptide or protein having a binding domain which is, or is substantially homologous to, an antibody binding domain. Examples of antibodies are the immunoglobulin isotypes and their isotypic subclasses; fragments which comprise an antigen binding domain such as Fab, scFv, Fv, dAb, Fd; and diabodies.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al, Proc. Natl. Acad. Sci. USA 90 6444-6448, 1993). Fv, scFv or diabody molecules may be stabilised by the incorporation of disulphide bridges linking the VH and VL domains (Y. Reiter et al. Nature Biotech 14 1239-1245 1996). Minibodies comprising an scFv joined to a CH3 domain may also be made (S. Hu et al, Cancer Res. 56 3055-3061 1996).

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804).

### Antigen Binding Domain

This describes the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains (e.g. a so-called Fd antibody fragment consisting of a VH domain). Preferably, an antigen binding domain comprises an antibody light chain variable region (VL) and an antibody heavy chain variable region (VH).

### Specific

This may be used to refer to the situation in which one member of a specific binding pair will not show any significant binding to molecules other than its specific binding partner(s). For example, an antibody specific for the ED-A isoform of fibronectin may show little or no binding to other isoforms of fibronectin. Similarly, an antibody specific for the ED-A domain of fibronectin may show little or no binding to other domains of fibronectin. The term is also applicable where e.g. an antigen binding domain is specific for a particular epitope which is carried by a number of antigens, in which case the specific binding member carrying the antigen binding domain will be able to bind to the various antigens carrying the epitope.

### Comprise

This is generally used in the sense of include, that is to say permitting the presence of one or more features or components. By “substantially as set out” it is meant that the relevant CDR or VH or VL domain of the invention will be either identical or highly similar to the specified regions of which the sequence is set out herein. By “highly similar” it is contemplated that from 1 to 5, preferably from 1 to 4 such as 1 to 3 or 1 or 2, or 3 or 4, substitutions may be made in the CDR and/or VH or VL domain.

The structure for carrying a CDR of the invention will generally be that of an antibody heavy or light chain sequence or substantial portion thereof in which the CDR is located at a location corresponding to the CDR of naturally occurring VH and VL antibody variable domains encoded by rearranged immunoglobulin genes. The structures and locations of immunoglobulin variable domains and CDRs may be determined by reference to (Kabat, E. A. et al, Sequences of Proteins of Immunological Interest. 4th Edition. US Department of Health and Human Services. 1987, and updates thereof, now available on the Internet (http://immuno.bme.nwu.edu)).

### Fibronectin

Fibronectin is an antigen subject to alternative splicing, and a number of alternative isoforms of fibronectin are known. Extra Domain-A (EDA or ED-A) is also known as ED, extra type III repeat A (EIIIA) or EDI. The sequence of human ED-A has been published by Kornblihtt et al. (1984), Nucleic Acids Res. 12, 5853-5868 and Paolella et al. (1988), Nucleic Acids Res. 16, 3545-3557. The sequence of human ED-A is also available on the SwissProt database as amino acids 1631-1720 (Fibronectin type-III 12; extra domain 2) of the amino acid sequence deposited under accession number PO2751. The sequence of mouse ED-A is available on the SwissProt database as amino acids 1721-1810 (Fibronectin type-III 13; extra domain 2) of the amino acid sequence deposited under accession number P11276.

The ED-A isoform of fibronectin (A-FN) contains the Extra Domain-A (ED-A). The sequence of the human A-FN can be deduced from the corresponding human fibronectin precursor sequence which is available on the SwissProt database under accession number PO2751. The sequence of the mouse A-FN can be deduced from the corresponding mouse fibronectin precursor sequence which is available on the SwissProt database under accession number P11276. The A-FN may be the human ED-A isoform of fibronectin. The ED-A may be the Extra Domain-A of human fibronectin.

ED-A is a 90 amino acid sequence which is inserted into fibronectin (FN) by alternative splicing and is located between domain 11 and 12 of FN (Borsi et al., 1987, J. Cell Biol., 104, 595-600). ED-A is mainly absent in the plasma form of FN but is abundant during embryogenesis, tissue remodelling, fibrosis, cardiac transplantation and solid tumour growth.

Various further aspects and embodiments of the present invention will be apparent to those skilled in the art in view of the present disclosure. All documents and database entries mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### EXPERIMENTAL

### Materials and Methods

### Cell Lines

For the combination therapy, the human renal cancer cell line Caki-1 (clear cell carcinoma from kidney; ATCC) was cultured in DMEM containing 10% fetal bovine serum and 2 mM L-glutamine (all Invitrogen).

### Animals

Female BALE/c nude mice were obtained from Charles River Laboratories. All experiments were performed according to Swiss regulations and under a project license granted by the Veterinäramt des Kantons Zürich.

Antibodies and Antibody Conjugates

F8 is a human monoclonal scFv antibody fragment specific to the alternatively spliced EDA domain of fibronectin and has been previously described (Villa et al., 2008).

The immunocytokine F8-IL2 was generated by cloning of F8 in a diabody format to human mature Interleukin-2, obtained from cDNA of human lymphocytes. The V_{H} and V_{L} domain of F8 were combined by a 5 aa linker (GGSGG) and fused via a flexible (S₄G)₃-linker to mature human IL2 (aa 21-153). F8-IL2 was produced in stable transfected CHO—S cells (Invitrogen) and purified by affinity chromatography on Protein A resins yielding a 95% pure protein running as homodimer in size exclusion analysis.

### Therapeutic Agents

Sorafenib tosylate (Nexavar, Bayer Schering) and sunitinib malate (Sutent, Pfizer) were obtained in the commercially available forms of 200 mg tablets and 50 mg capsules, respectively. The total tablet weight of sorafenib is 350 mg and an index of 1.75 was used to keep the molarity in terms of active compound. Sorafenib tablets were pulverized with mortar and pestle and kept from light in a desiccator. For in vivo administration, pulverized sorafenib was dissolved in a solution of CremophorEL:Ethanol:H₂O (12.5%:12.5%:75%). The content of a sunitinib malate capsule was 167 mg and a correction factor of 3.3 was used for molarity adjustment. Sunitinib powder was stored in the dark in a dessicator and dissolved in 0.5% carboxymethylcellulose/0.4% Tween80/1.8% NaCl/0.9% benzyl alcohol for in vivo studies. Human interferon alpha 2b (IntronA, Schering-Plough) was obtained from the pharmacy as 10 Mio IU ampule and recombinant murine interferon alpha 1 (1*10⁵ U) was purchased from PBL interferon source.

### Tumour Mouse Model

Tumor-bearing mice were obtained by subcutaneous (s.c.) injection of 1×10⁷ Caki-1 cells in the left flank of 10-12 week-old female BALB/c nude mice at day 0. When tumors reached a size of 80-100 mm³, mice were grouped (n=5-8) to obtain uniformity among the groups and started to treat (1^{st} treatment cycle).

For the single treatment, 20 μg of F8-IL2 were injected intravenously (i.v.) into the lateral tail vein in 100 μl PBS three times every 3^{rd} day, 60 mg/kg sorafenib or 60 mg/kg sunitinib were given orally for 7 consecutive days, or a mixture of huIFNa2b/muIFNa1 at 10.000 IU/10.000 IU in 100 μl PBS was administered s.c. in the distant flank three times every 3^{rd} day.

For the combination treatment, administration of 20 μg F8-IL2 (i.v., 3× every 3^{rd} day)) was combined with the administration of 60 mg/kg sorafenib (p.o., 7×, daily), 60 mg/kg sunitinib (p.o., 7×, daily) or 10.000 IU/10.000 IU of huIFNa2b/muIFNa1 (s.c., 3× every 3^{rd} day). A control group received 100 μl of PBS i.v. in the schedule of F8-IL2 administration. An identical 2^{nd} treatment cycle was started 20 days after a treatment-free interval.

Mice were monitored daily and tumor volumes were measured three times per week with a digital caliper using the following formula: volume=length×width²×0.5. Animals were sacrificed when tumor volumes reached >2000 mm³. Tumor volumes are expressed as mean±SE (standard error). Tumor growth curves were stopped when the first tumor per treatment group reached >2000 mm³.

### Results

### F8-IL2 and Sorafenib

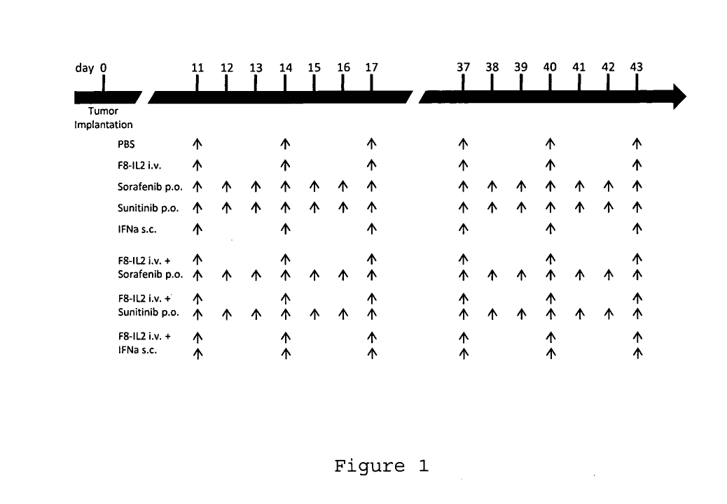

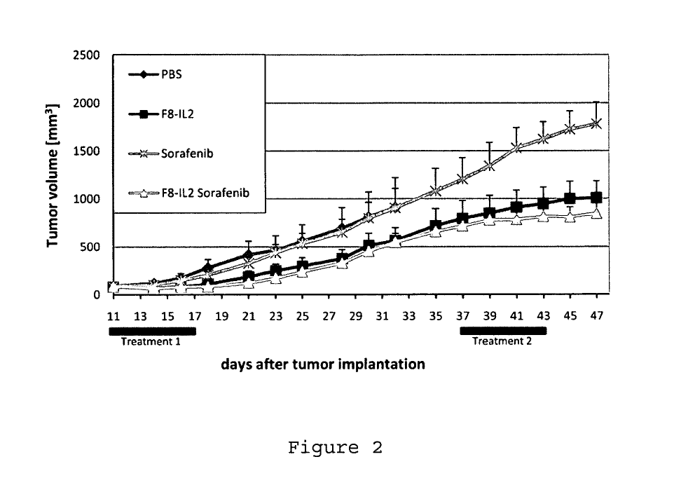

Comparison of the tumour volume (or tumour burden) in mice injected subcutaneously with Caki-1 cells, and treated either with F8-IL2 or PBS (control) according to the treatment schedule shown in FIG. 1, showed that treatment F8-IL2 significantly reduced the tumour burden in these mice (FIG. 2). In contrast, treatment with Sorafenib alone had little or no effect on tumour burden, which was similar to that observed in control mice “treated” with PBS (FIG. 2). When mice were treated with F8-IL2 in combination with Sorafenib no statistically significant change in the tumour burden was observed compared with the tumour burden in mice treated with F8-IL2 alone.

F8-IL2 and Interferon Alpha

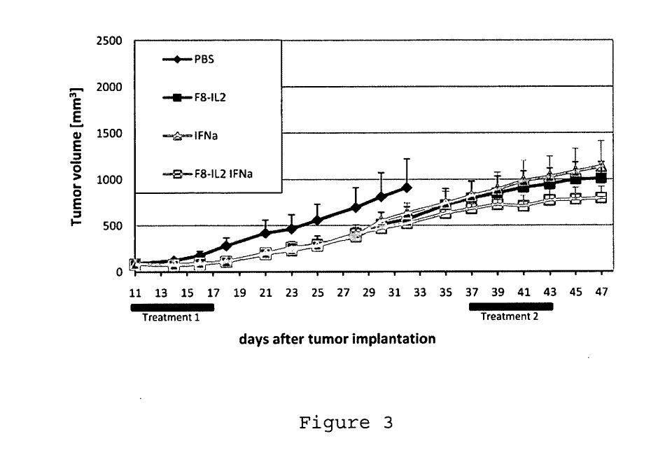

The tumour volume in mice treated with F8-IL2, interferon alpha (IFNa), or F8-IL2 and interferon alpha, was also compared to the tumour volume in control mice “treated” with PBS (FIG. 3). Treatment with either F8-IL2, or interferon alpha, alone showed a similar reduction in the tumour burden compared with the tumour burden observed in control mice (FIG. 3). When mice were treated with F8-IL2 in combination with interferon alpha no statistically significant change in the tumour burden was observed compared with the tumour burden in mice treated with either F8-IL2, or interferon alpha, alone (FIG. 3).

F8-IL2 and Sunitinib

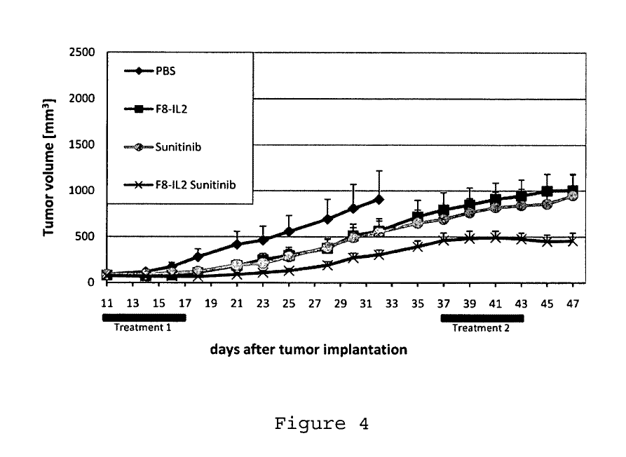

When the tumour volume in mice treated with F8-IL2, sunitinib, or F8-IL2 and sunitinib, was compared to the tumour volume in control mice “treated” with PBS the following effect was seen (FIG. 4). Treatment with either sunitinib or F8-IL2 alone resulted in a similar reduction in the tumour burden (FIG. 4). However, when mice were treated with F8-IL2 in combination with sunitinib, a much greater reduction in the tumour burden was observed than in mice treated with either F8-IL2, or sunitinib alone (FIG. 4).

### CONCLUSION

Treatment of mice with F8-IL2 in combination with either Sorafenib or interferon alpha showed no statistically significant improvement in the tumour burden in these mice compared with the tumour burden in mice treated with either F8-IL2, or interferon alpha alone (FIGS. 2 and 3).

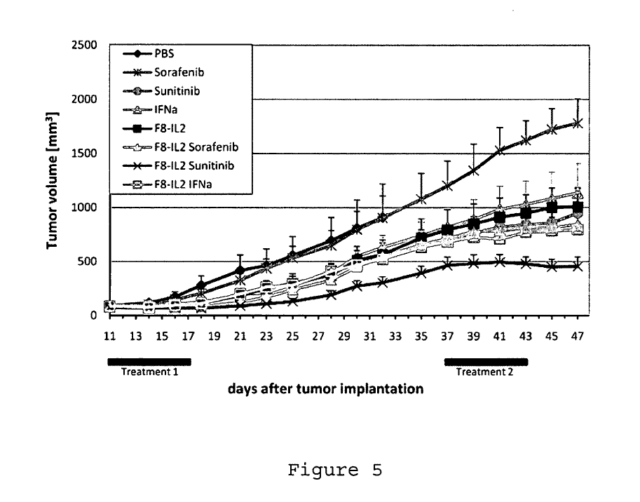

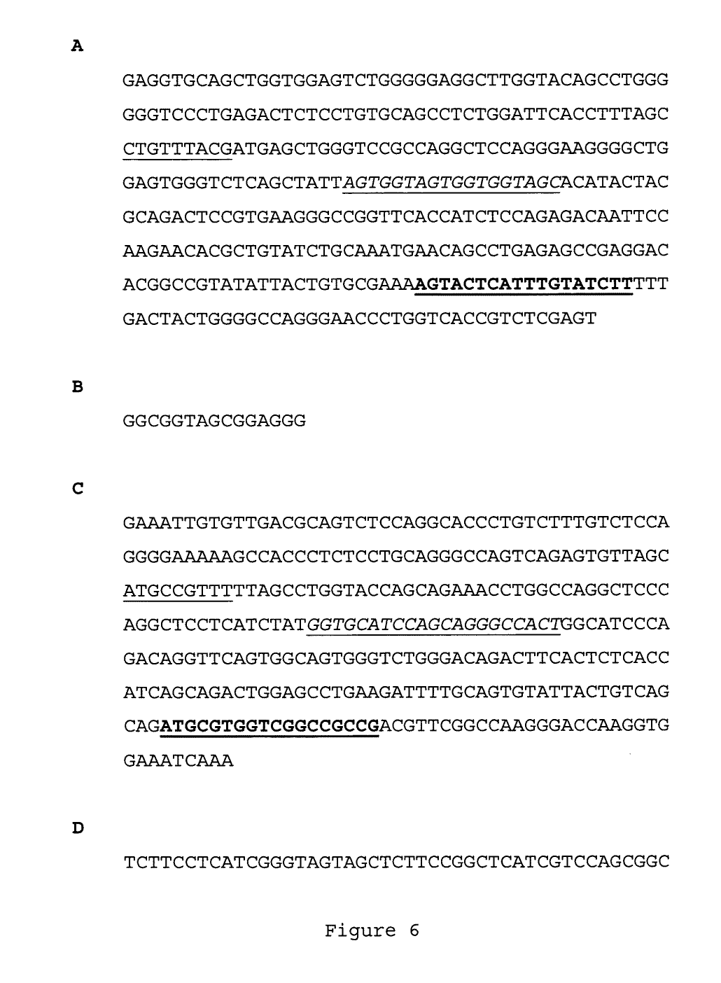

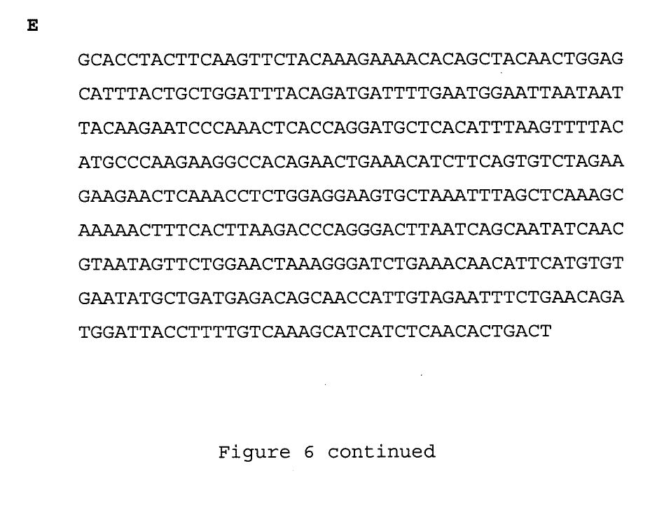

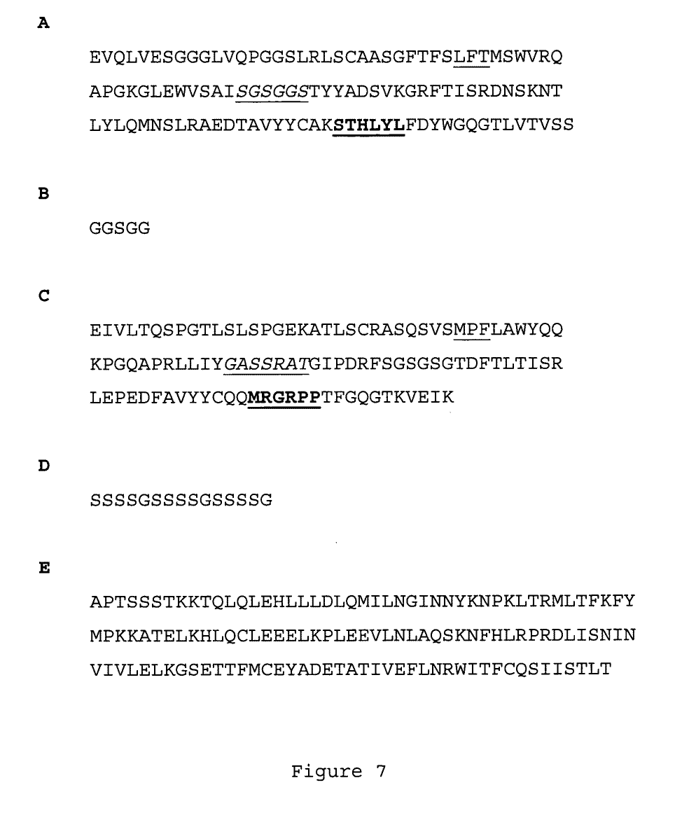

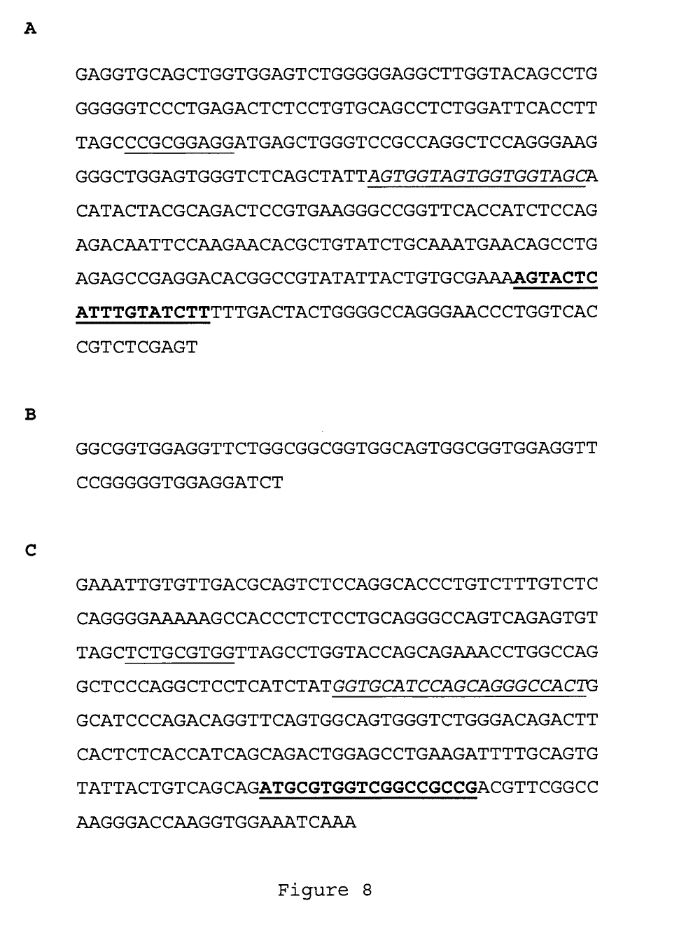

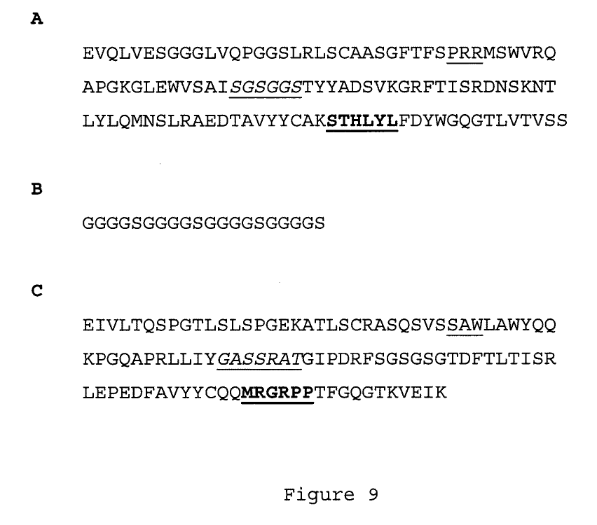

However, surprisingly, when mice were treated with F8-IL2 in combination with sunitinib the reduction in tumour burden observed was much greater than that observed with any other form of treatment (FIG. 5). In addition, the reduction in tumour burden observed when these agents were administered in combination was much greater than would have been expected based on levels of reduction in tumour burden observed in mice treated with either F8-IL2 or sunitinib alone. This is evident for example from the fact that treating mice with e.g. F8-IL2 and interferon alpha did not have the same synergistic effect (FIG. 3).

Thus, conjugate F8-IL2 exhibits an unexpected synergy with anti-cancer compound sunitinib in the treatment of cancer and suggests that treatment of human cancers with sunitinib may also be improved by administering sunitinib in combination with F8-IL2. Such a combined treatment may, for example, result in a greater reduction in the tumour burden of the individual than can be achieved through treatment with sunitinib alone.

TABLE 1
Nucleotide and amino acid sequences of the
heavy chain (VH) and light chain (VL)
CDR1s of anti-ED-A antibodies

| Antibody | CDR1 (VH) | CDR1 (VL) |
|---|---|---|
| H1 | CCG CGG AGG | TCT GCG TGG |
| | P R R (SEQ ID NO: 3) | S A W (SEQ ID NO: 6) |
| B2 | GCG GCT AAG | GTG GCT TTT |
| | A A K (SEQ ID NO: 23) | V A F (SEQ ID NO: 26) |
| C5 | CCG ATT ACT | TTG CAT TTT |
| | P I T (SEQ ID NO: 43) | L H F (SEQ ID NO: 46) |
| D5 | GTG ATG AAG | AAT GCT TTT |
| | V M K (SEQ ID NO: 53) | N A F (SEQ ID NO: 56) |
| E5 | ACT GGT TCT | CTT GCG CAT |
| | T G S (SEQ ID NO: 63) | L A H (SEQ ID NO: 66) |
| C8 | CTT CAG ACT | CTT CCT TTT |
| | L Q T (SEQ ID NO: 73) | L P F (SEQ ID NO: 76) |
| F8 | CTG TTT ACG | ATG CCG TTT |
| | L F T (SEQ ID NO: 83) | M P F (SEQ ID NO: 86) |
| F1 | TAG GCG CGT | GCG CCT TTT |
| | Q(Amber) A R (SEQ ID NO: 93) | A P F (SEQ ID NO: 96) |
| B7 | CAT TTT GAT | CTG GCT TTT |
| | H F D (SEQ ID NO: 103) | L A F (SEQ ID NO: 106) |
| E8 | GAT ATG CAT | TCG TCT TTT |
| | D M H (SEQ ID NO: 113) | S S F (SEQ ID NO: 116) |
| G9 | CAT ATG CAG | ACT GCT TTT |
| | H M Q (SEQ ID NO: 33) | T A F (SEQ ID NO: 36) |

### REFERENCES

Balza et al. (1988), FEBS Lett., 228: 42-44.
Borsi et al. (1987), J. Cell. Biol., 104, 595-600.
Borsi et al. (1990), FEBS Lett., 261: 175-178.
Borsi et al. (1995), J. Biol. Chem., 270: 6243-6245.
Carnemolla et al. (1989), J. Cell. Biol., 108: 1139-1148.
Jacobs et al. (2002), Hum. Pathol., 33, 29-38.
Lohi et al. (1995), Int. J. Cancer, 63, 442-449.
Matsumoto et al. (1999), Jpn. J. Cancer Res., 90, 320-325.
Oyama et al. (1989), J. Biol. Chem., 264, 10331-10334.
Tavian et al. (1994), Int. J. Cancer, 56, 820-825.
Villa A et al. Int. J. Cancer. 2008 Jun. 1; 122(11):2405-13.

1-14. (canceled)

15. A kit comprising sunitinib and an antibody-IL2 conjugate comprising interleukin 2 (IL2) conjugated to an antibody which specifically binds to the ED-A isoform of fibronectin, wherein the antibody-IL2 conjugate and sunitinib are for treatment of cancer.

16. A method of treating cancer comprising administering sunitinib and an antibody-interleukin 2 (IL2) conjugate to an individual in need thereof, wherein the antibody-IL2 conjugate comprises IL2 conjugated to an antibody which specifically binds the Extra Domain-A (ED-A) isoform of fibronectin.

17. A method according to claim 16, wherein the antibody specifically binds the ED-A of fibronectin.

18. A method according to claim 16, wherein the antibody comprises an antibody antigen binding site comprising a VH domain and a VL domain, the VH domain comprising a VH CDR1 of SEQ ID NO: 83, a VH CDR2 of SEQ ID NO. 4, and a VH CDR3 of SEQ ID NO. 5, and the VL domain comprising a VL CDR1 of SEQ ID NO: 86, a VL CDR2 of SEQ ID NO. 7, and a VL CDR3 of SEQ ID NO. 8.

19. A method according to claim 16, wherein the antibody comprises an antibody antigen binding site comprising the F8 VH domain of SEQ ID NO. 81 and the F8 VL domain of SEQ ID NO. 82.

20. A method according to claim 16, wherein the antibody is a single chain Fv.

21. A method according to claim 16, wherein the cancer is kidney cancer.

22. A method according to claim 17, wherein the antibody comprises an antibody antigen binding site comprising a VH domain and a VL domain, the VH domain comprising a VH CDR1 of SEQ ID NO: 83, a VH CDR2 of SEQ ID NO. 4, and a VH CDR3 of SEQ ID NO. 5, and the VL domain comprising a VL CDR1 of SEQ ID NO 86, a VL CDR2 of SEQ ID NO 7, and a VL CDR3 of SEQ ID NO. 8.

23. A method according to claim 17, wherein the antibody comprises an antibody antigen binding site comprising the F8 VH domain of SEQ ID NO. 81 and the F8 VL domain of SEQ ID NO. 82.

24. A method according to claim 17, wherein the antibody is a single chain Fv.

25. A method according to claim 17, wherein the cancer is kidney cancer.

26. A method according to claim 18, wherein the antibody comprises an antibody antigen binding site comprising the F8 VH domain of SEQ ID NO. 81 and the F8 VL domain of SEQ ID NO. 82.

27. A method according to claim 18, wherein the antibody is a single chain Fv.

28. A method according to claim 18, wherein the cancer is kidney cancer.

29. A method according to claim 19, wherein the antibody is a single chain Fv.

30. A method according to claim 19, wherein the cancer is kidney cancer.

31. A method according to claim 20, wherein the cancer is kidney cancer.
